(19) United States
(12) Patent Application Publication
(10) Pub. No.: US 2010/0316623 A1
(43) Pub. Date: Dec. 16, 2010

(54) PHENYLALANINE HYDROXYLASE FUSION PROTEIN AND METHODS FOR TREATING PHENYLKETONURIA

(76) Inventors: Andrew TURNER, Durham, NC (US); Homayoun SADEGHI, Hillsborough, NC (US)

Correspondence Address: COOLEY LLP;ATTN: Patent Group, Suite 1100, 777 - 6th Street, NW, WASHINGTON, DC, 20001, US

(21) Appl. No.: 12/766,193

(22) Filed: Apr. 23, 2010

Related U.S. Application Data
(60) Provisional application No. 61/171,985, filed on Apr. 23, 2009.
(60) Provisional application No. 61/247,619, filed on Oct. 1, 2009.

(51) Int. Cl. A61K 38/44; C12N 9/02; C07H 21/04; C12N 15/63; C12N 5/10

(52) U.S. Cl. 424/94.4; 435/189; 536/23.2; 435/320.1; 435/325

(57) ABSTRACT

The present invention provides Phenylalanine Hydroxylase (PAH) fusion proteins and pharmaceutical compositions comprising the same, as well as encoding polynucleotides and vectors, and methods for treating hyperphenylalaninemia, including PKU, by enzyme replacement therapy. The fusion proteins have phenylalanine hydroxylase activity when administered: and have an increased half-life or persistence in circulation, as compared to unfused counterparts. The PAH fusion proteins are suitable for enzyme replacement therapy in PKU patients by converting phenylalanine in the circulation to tyrosine, thereby controlling phenylalanine levels.

### CROSS REFERENCE TO RELATED APPLICATIONS

This application claims priority to U.S. Provisional Application No. 61/171,985, filed Apr. 23, 2009, and U.S. Provisional Application No. 61/247,619, filed Oct. 1, 2009, each of which is hereby incorporated by reference in its entirety.

### FIELD OF THE INVENTION

The present invention relates the treatment of disease characterized by elevated levels of phenylalanine, and in particular phenylketonuria (PKU). The present invention relates to enzyme replacement therapy for PKU with phenylalanine hydroxylase fusion proteins.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: PHAS_{—}018_{—}02US_SeqList_ST25.txt, date recorded: April 23, 2010, file size 29 kilobytes).

### BACKGROUND

Phenylketonuria (PKU), or its less severe form hyperphenylalaninemia, are metabolic disorders in which the absence of sufficient phenylalanine hydroxylase activity results in the accumulation of phenylalanine in the body. When untreated or uncontrolled, the accumulation of phenylalanine can be toxic, potentially resulting in neurological deficits, cognitive disorders including mental retardation, psychiatric disorders, and physical handicap.

Phenylalanine Hydroxylase (PAH) is the enzyme that converts phenylalanine, an essential amino acid supplied by the diet, to the amino acid tyrosine. PAH acts intracellularly, and mainly in the liver. PAH is a complex enzyme having a homotetrameric structure, and requires several cofactors for activity, including an active-site bound Fe²⁺, tetrahydrobiopterin (BH₄), and molecular oxygen. Over 500 mutations have been documented as resulting in a dysfunction in phenylalanine metabolism, and 99% of the mutant alleles map to the PAH gene.

PKU is often controlled by monitoring the dietary intake of phenylalanine by a semi-synthetic diet that is low in phenylalanine. Since it is difficult to carefully control phenylalanine intake indefinitely, other strategies for controlling accumulation of phenylalanine levels in PKU patients have been developed. These include KUVAN™ (sapropterin hydrochloride), which is a synthetic version of the PAH cofactor BH₄. Unfortunately, 50 to 80% of PKU patients do not respond to KUVAN™, and thus other strategies such as enzyme therapy with PAH or Phenylalanine Ammonia Lyase (PAL) from plant, bacteria, or yeast have been proposed. See Gamez et al., Toward PKU Enzyme Replacement Therapy: PEGylation with Activity Retention for Three Forms of Recombinant Phenylalanine Hydroxylase, Molecular Therapy 9(1):124-129 (2004); and Kim et al., Trends in Enzyme Therapy for Phenylketonuria, Molecular Therapy. 10(2):220-224 (2004). PAL converts phenylalanine to trans-cinnamic acid, a harmless byproduct, and is not dependent on BH₄ cofactor. PAL can be highly immunogenic and rapidly removed from circulation, complicating its potential for enzyme replacement therapy. Further, PAL therapy may also require dietary Tyrosine supplementation, as Tyrosine is not a product of the reaction.

Alternative strategies for treating PKU or hyperphenylalaninemia are needed, including enzyme replacement strategies.

### SUMMARY OF THE INVENTION

The present invention provides Phenylalanine Hydroxylase (PAH) fusion proteins and pharmaceutical compositions comprising the same, as well as encoding polynucleotides and vectors, and methods for treating hyperphenylalaninemia, including PKU, by enzyme replacement therapy. The fusion proteins have phenylalanine hydroxylase activity when administered (e.g., by injection), and have an increased half-life or persistence in circulation, as compared to unfused counterparts. The PAH fusion proteins are suitable for enzyme replacement therapy in PKU patients by converting phenylalanine in the circulation to tyrosine, thereby controlling phenylalanine levels.

In one aspect, the invention provides fusion proteins between PAH and heterologous amino acid sequences that extend half-life of the molecule in the circulation. The invention further provides pharmaceutical compositions comprising the fusion proteins. The fusion proteins comprise the catalytic domain of PAH, for example, amino acid residues 103-428 of PAH, and comprise a heterologous amino acid sequence, such as an Elastin-Like-Protein component or domain as described herein. The PAH fusion protein is able to form active enzyme, and in some embodiments, is catalytically active with endogenous BH₄ cofactor present in the circulation (e.g., without cofactor supplementation). In some embodiments, the PAH fusion protein has a specific activity similar to the unfused PAH counterpart, as determined by an assay described herein. Further still, the PAH fusion protein persists in the circulation after administration, allowing for less frequent administrations than alternative enzyme replacement therapies (e.g., unfused or PEGylated PAH, or unfused or PEGylated PAL).

The PAH fusion protein may be administered to patients by injection, for example, by subcutaneous injection. In some embodiments employing ELP fusion sequences, the fusion protein may be designed to form a drug depot upon injection through a phase transition of the ELP component at body temperature to a gel-like form. The transitioned fusion depot will gradually release fusion protein over time to provide a sustained release of PAH fusion protein in the circulation.

In a second aspect, the present invention provides polynucleotides and vectors encoding the PAH fusion proteins, and methods of making and purifying the fusion proteins. The polynucleotides and vectors provide for recombinantly produced PAH fusions, which in some embodiments may be conveniently recovered in purified form by inverse temperature cycling as described herein. The encoding polynucleotides and vectors allow for various fusion sequences to be conveniently inserted and positioned relative to PAH sequences, to support enzyme structure and activity, as well as to support ELP transition properties and half-life of the fusion protein in the circulation for example. The polynucleotides and vectors further provide for the convenient addition of other components such as cleavable linkers and targeting elements as described herein.

In a third aspect, the invention provides a method for treating hyperphenylalaninemia or PKU by enzyme replacement. The method generally comprises administering the PAH fusion protein to a patient in need. The fusion protein may be administered by injection, for example, by subcutaneous injection, or may be administered orally. When administered parenterally, the PAH fusion protein lowers phenylalanine levels in the circulation, for example, using only endogenous BH₄ cofactor in some embodiments. The PAH fusion protein may be designed as a soluble fusion protein, or may be designed to form a drug depot upon injection as described herein. In various embodiments, the PAH fusion protein may be administered at a frequency of about 1 to 3 times per day, about 1 to 2 times per week, or in other embodiments, 1 to 4 times per month, to control phenylalanine levels over time.

### DESCRIPTION OF THE FIGURES

FIG. 1 shows a coding sequence (SEQ ID NO:13) and amino acid sequence (SEQ ID NO:14) comprising the core catalytic domain of PAH (amino acids 103-428 of PAH), as amplified with PCR primers for convenient cloning of the insert into ELP-containing vectors. The insert will result in the first VPGXG repeat (SEQ ID NO: 3) being truncated as GVG. PCR primers used for amplification and cloning (P0051 and P0052, SEQ ID NOS:15 and 16, respectively) are also shown. Primers are designed with EcoRI and PflMI restriction sites for cloning of the resulting insert.

FIG. 2 illustrates the plasmid cloning vector designated pPB0996, which encodes 120 repeats of the ELP1 pentamer (VPGXG, SEQ ID NO: 3) downstream of the EcoRI and PflMI cloning sites.

FIG. 3 illustrates the plasmid designated pPB0998, which encodes 120 repeats of the ELP1 pentamer (VPGXG, SEQ ID NO: 3) in frame with the PAH catalytic domain (cdPAH). The PAH-ELP encoding insert may be recovered by digestion of pPB0998 with Xbal and BglI.

FIG. 4 illustrates the plasmid designated pPB0913, which is a PET expression vector having Xbal and BglI cloning sites.

FIG. 5 illustrates the pET expression vector designated pPB0999, having the cdPAH-ELP fusion insert cloned into the Xbal and BglI sites.

FIG. 6 shows a coding sequence (SEQ ID NO:17) and amino acid sequence (SEQ ID NO:18) comprising the core catalytic domain of PAH (amino acids 103-428), as amplified with two sets of PCR primers and assembled. PCR primers used for amplification (P0053, P0054, P0055, and P0056; SEQ ID NOS:19 to 22, respectively) are also shown. The insert is designed for isolation by BglI/HindIII restriction enzymes.

FIG. 7 illustrates the plasmid designated pPB1000, which is pPB0996 with the BglI/HindIII fragment of FIG. 6 cloned in frame with ELP1-120 as shown.

FIG. 8 illustrates the plasmid designated pPB1001, which is modified at the 5′ end of the expression cassette (relative to pPB1000) to provide the start MVPGVG (SEQ ID NO: 31). Specifically, a linker was inserted at the EcoRI/PflMI site of pPB1000.

FIG. 9 illustrates the pET expression vector designated pPB1002, having the ELP1-120-cdPAH fusion inserted at the Xbal/Nhel cloning site.

FIG. 10 illustrates the cdPAH coding sequence for N-terminal fusion, optimized for E. coli expression (SEQ ID NO:28). The encoded amino acid sequence is SEQ ID NO:14.

FIG. 11 illustrates the cdPAH coding sequence for C-terminal fusion, optimized for E. coli expression (SEQ ID NO:29). The encoded amino acid sequence is SEQ ID NO:30.

FIG. 12 illustrates the expression in E. coli and subsequent purification of PAH (103-428)-ELP1-120 (designated PB0999). The expected molecular weight of 85 kDa is shown by SDS-PAGE (denaturing, non-reducing).

FIG. 13 shows that PB0999 is active in an assay for converting phenylalanine to tryrosine, as determined by OD450 nm.

FIG. 14 shows a kinetic assay for PAH-ELP activity. Enzyme activity was measured by phenylalanine-dependent oxidation of NADH at 340 nm. See Macdonald et al., (1990), PNAS 87, 1965-1967.

FIG. 15A illustrates the conversion of phenylalanine to tyrosine by PAH, with BH₄ and O₂ as cofactors. As shown in FIG. 15B, PAH-ELP converts phenylalanine to tyrosine, as determined by an increase in OD₂₇₅. The PAH-ELP comprises PAH(103-428) with 120 pentamer ELP repeats, and exhibits a specific activity of 878 nmol tyrosine/min·mg.

FIG. 16 shows that tyrosine production by PAH-ELP is dose dependent.

FIG. 17 shows the conversion of phenylalanine to tyrosine by PAH-ELP (3 μg) versus a no PAH control, as determined by RP-HPLC (Shimadzu C18 column).

FIG. 18 shows the effect of PAH-ELP on blood phenylalanine levels in mice as measured by RP-HPLC.

FIG. 19 shows the average levels of blood phenylalanine in mice following administration of either PAH-ELP or control buffer as measured by RP-HPLC.

### DETAILED DESCRIPTION OF INVENTION

The present invention provides Phenylalanine Hydroxylase (PAH) fusion proteins and pharmaceutical compositions comprising the same, as well as encoding polynucleotides and vectors, and methods for treating hyperphenylalaninemia, including PKU, by enzyme replacement therapy. The fusion proteins have Phenylalanine Hydroxylase activity when administered by injection, and in some embodiments may persist in the circulation to provide sustained biological activity.

The fusion proteins of the invention comprise the catalytic core of human PAH and a heterologous amino acid sequence. For example, the heterologous sequence may be Elastin-Like Protein (ELP) as described herein. The PAH fusion protein is able to form active enzyme, and in some embodiments, is catalytically active with endogenous BH₄ cofactor present in the circulation.

### Phenylalanine Hydroxylase

The invention provides fusion proteins comprising the catalytic core of PAH. The nucleotide sequence of the PAH gene is known, as well as the encoded amino acid sequence (Gene ID 5053). The nucleotide sequence encoding a complete open reading frame for human Phenylalanine Hydroxylase, as well as the complete amino acid sequence, are provided herein as SEQ ID NOS: 23 and 24, respectively.

The fusion protein of the invention comprises the catalytic domain of PAH. The full length human PAH enzyme comprises a central catalytic domain that contains sites for substrate, iron and BH₄ cofactor binding; an N-terminal region with regulatory properties; and a C-terminal domain involved with inter-subunit binding (Hufton et al., Structure and function of the aromatic amino acid hydroxylases, Biochem J, 1995, 311:353-366; Waters et al., In vitro expression analysis of mutations in phenylalanine hydroxylase: linking genotype to phenotype and structure to function, Hum. Mutat, 1998, 11:4-17). The wild-type Phenylalanine Hydroxylase is a tetramer composed of four monomers (4 identical subunits).

The N-terminal regulatory domain of PAH spans the approximately 115 amino acids nearest the amino terminal of each subunit. The catalytic domain is composed of the next approximately 300 amino acids, and is responsible for all of the catalytic activity of the enzyme. The C-terminal tetramerization domain consists of the remaining amino acids, and through the formation of a coiled-coil arrangement of amino acids, holds the tetrameric structure of the holoenzyme together with a leucine zipper.

The compounds, compositions, and methods of the invention employ the catalytic domain of PAH fused to a heterologous sequence, such as ELP. Thus, the fusion protein may comprise a PAH amino acid sequence beginning at an amino acid within residues 90 to 115 of human PAH. For example, the PAH domain of the fusion protein may begin at about amino acid 80, 95, 100, 103, 110, or 115 of the wild type PAH sequence. The fusion protein may comprise the PAH amino acid residues starting from within the range of residues 90 to 115 of PAH, through an amino acid in the range of residues 400 to 450 of the wild type sequence. For example, the PAH domain of the fusion protein may terminate with or around amino acid 400, 405, 415, 420, 425, 428, or 430 of the wild type sequence. In certain embodiments, the fusion protein comprises, or consists essentially of, or consists of, amino acid residues 103-428 of PAH as described in Knappskog et al., Structure/function relationships in human phenylalanine hydroxylase. Eur. J. Biochem. 1996, 242:813-821. Amino acids 103 to 428 of wild type PAH is referred to herein as SEQ ID NO:25. The fusion proteins may comprise additional amino acids adjacent to positions 103 and 428 of the wild type enzyme (e.g., such as from 1 to 10, or from 1 to 5 amino acids adjacent on the N-terminal and/or C-terminal side), or modifications at these adjacent positions, as described below.

When fused to a heterologous protein, the catalytic domain of PAH is able to form active, dimeric enzyme, utilizing iron, BH₄ and molecular oxygen as cofactors for the conversion of phenylalanine to tyrosine. Further, when fused (e.g., with ELP), the enzyme substantially retains its specific activity.

The invention may employ various insertions, deletions, and/or substitutions within the PAH component, so long as the activity for converting phenylalanine to tyrosine is substantially maintained. The crystal structure of the catalytic domain has been described, and can be employed to guide such substitutions, insertions and/or deletions in the PAH sequence. See, e.g., Andersen et al., Crystal Structure of the Ternary Complex of the Catalytic Domain of Human Phenylalanine Hydroxylase With Tetrahydrobiopterin and 3-(2-Thienyl)-L-alanine, and its Implications for the Mechanism of Catalysis and Substrate Activation. J. Mol. Biol. 320:1095-1108 (2002). For example, the following amino acids are described as being in the active-site crevice or involved in binding of pterin cofactor: Tyr138, Gly247, Leu248, Ser251, Phe254, Leu255, His264, Glu286, Ala322, Tyr325, Glu330, Arg270, Tyr277, Thr278, Pro281, His285, Trp326, Phe331, Gly346, Ser349, Ser350, and Glu353.

Further, mutational analysis of PAH has been conducted, and such analyses may likewise guide appropriate modification of the enzyme, where desired, in accordance with the invention. Such studies include Waters et al., In vitro expression analysis of mutations in phenylalanine hydroxylase: linking genotype to phenotype and structure to function, Hum. Mutat, 1998, 11:4-17); and Erlandsen et al., Crystal Structure and Site-Specific Mutagenesis of Pterin-Bound Human Phenylalanine Hydroxylase, Biochemistry 39:2208-2217 (2000).

Thus, in certain embodiments, the catalytic domain of PAH comprises, consists essentially of, or consists of SEQ ID NO:25 or a functional analog thereof. Such functional analogs may contain from 1 to 10 amino acid insertions, deletions, and/or substitutions (collectively) with respect to the native sequence (e.g., SEQ ID NO:25) and in each case retaining or enhancing the activity of the enzyme. For example, the functional analog of the PAH catalytic domain may have from 1 to about 10, such as 3, 4, or 5-insertions, deletions and/or substitutions (collectively) with respect to SEQ ID NO:25, and in each case retaining the activity of the enzyme. Such activity may be confirmed or assayed by determining, or by determining the rate of, phenylalanine conversion to tyrosine. Exemplary assays for confirming PAH enzyme activity are described herein. In these or other embodiments, the PAH catalytic domain has at least about 70%, 75%, 80%, 85%, 90%, or 95% identity with the native sequence (SEQ ID NO:25). The determination of sequence identity between two sequences (e.g., between a native sequence and a functional analog) can be accomplished using any alignment tool, including Tatusova et al., Blast 2 sequences—a new tool for comparing protein and nucleotide sequences, FEMS Microbiol Lett. 174:247-250 (1999).

### Heterologous Fusion Sequences and Elastin-Like-Protein

The fusion protein of the invention comprises one or more heterologous sequences. Such sequences in certain embodiments may be mammalian sequences, such as albumin, transferrin, or antibody sequences. Such sequences are described in See U.S. Pat. No. 7,238,667 (particularly with respect to albumin conjugates), U.S. Pat. No. 7,176,278 (particularly with respect to transferrin conjugates), and U.S. Pat. No. 5,766,883, which are each hereby incorporated by reference in their entireties.

In certain embodiments, the heterologous sequence is an Elastin-Like-Protein sequence. The ELP sequence comprises or consists of structural peptide units or sequences that are related to, or derived from, the elastin protein. Such sequences are useful for improving the properties of PAH in one or more of bioavailability, therapeutically effective dose and/or administration frequency, enzymatic action, formulation compatibility, resistance to proteolysis, solubility, half-life or other measure of persistence in the body subsequent to administration, and/or rate of clearance from the body.

The ELP component is constructed from structural units of from three to about twenty amino acids, or in some embodiments, from four to ten amino acids, such as five or six amino acids. The length of the individual structural units, in a particular ELP component, may vary or may be uniform. In certain embodiments, the ELP component is constructed of a polytetra-, polypenta-, polyhexa-, polyhepta-, polyocta, and polynonapeptide motif of repeating structural units. Exemplary structural units include units defined by SEQ ID NOS: 1-12 (below), which may be employed as repeating structural units, including tandem-repeating units, or may be employed in some combination, to create an ELP effective for improving the properties of the therapeutic component. Thus, the ELP component may comprise or consist essentially of structural unit(s) selected from SEQ ID NOS: 1-12, as defined below.

The ELP component, comprising such structural units, may be of varying sizes. For example, the ELP component may comprise or consist essentially of from about 10 to about 500 structural units, or in certain embodiments about 25 to about 200 structural units, or in certain embodiments from about 50 to about 150 structural units, or from about 60 to about 120 structural units, including one or a combination of units defined by SEQ ID NOS: 1-12. Thus, the ELP component may have a length of from about 50 to about 2000 amino acid residues, or from about 100 to about 800 amino acid residues, or from about 200 to about 700 amino acid residues, or from about 400 to about 600 amino acid residues.

In some embodiments, the ELP component in the untransitioned state may have an extended, relatively unstructured and non-globular form, so as to escape kidney filtration. Thus, even in embodiments where the fusion protein has a molecular weight of less than the generally recognized cut-off for filtration through the kidney, such as less than about 60 kD, the molecule will persist in the body by at least 2-fold, 3-fold, 4-fold, 5-fold, 10-fold, 20-fold, or 100-fold longer than an uncoupled (e.g., unfused or unconjugated) PAH counterpart.

In these or other embodiments, the ELP component does not substantially or significantly impact the biological action of the PAH. Thus, the therapeutic agent of the invention exhibits a potency (enzymatic action) that is the same or similar to its unfused counterpart. For example, the therapeutic agent of the invention may exhibit a potency or level of enzymatic action (e.g., as tested in vitro or in vivo) of at least 40% of that exhibited by the unfused counterpart of the therapeutic agent in the same assay. In various embodiments, the therapeutic agent may exhibit a potency or level of enzymatic action (e.g., as tested in vitro or in vivo) of at least 50%, 60%, 75%, 80%, 90%, or more of that exhibited by the unfused counterpart. For example, enzymatic action may be determined in vitro by measuring enzyme conversion of phenylalanine to tyrosine as described herein. Any suitable measure of enzyme specific activity or kinetics may be employed in such comparisons.

In certain embodiments, the ELP component undergoes a reversible inverse phase transition. That is, the ELP components are structurally disordered and highly soluble in water below a transition temperature (Tt), but exhibit a sharp (2-3° C. range) disorder-to-order phase transition when the temperature is raised above the Tt, leading to desolvation and aggregation of the ELP components. For example, the ELP forms insoluble polymers, when reaching sufficient size, which can be readily removed and isolated from solution by centrifugation. Such phase transition is reversible, and isolated insoluble ELPs can be completely resolubilized in buffer solution when the temperature is returned below the Tt of the ELPs. Thus, the therapeutic agents of the invention can, in some embodiments, be separated from other contaminating proteins to high purity using inverse transition cycling procedures, e.g., utilizing the temperature-dependent solubility of the therapeutic agent, or salt addition to the medium. Successive inverse phase transition cycles can be used to obtain a high degree of purity. In addition to temperature and ionic strength, other environmental variables useful for modulating the inverse transition of the therapeutic agents include pH, the addition of inorganic and organic solutes and solvents, side-chain ionization or chemical modification, and pressure.

In certain embodiments, the ELP component does not undergo a reversible inverse phase transition, or does not undergo such a transition at a biologically relevant Tt, and thus the improvements in the biological and/or physiological properties of the molecule (as described elsewhere herein), may be entirely or substantially independent of any phase transition properties. Nevertheless, such phase transition properties may impart additional practical advantages, for example, in relation to the recovery and purification of such molecules.

In certain embodiments, the ELP component(s) may be formed of structural units, including but not limited to:
(a) the tetrapeptide Val-Pro-Gly-Gly, or VPGG (SEQ ID NO: 1);
(b) the tetrapeptide Ile-Pro-Gly-Gly, or IPGG (SEQ ID NO: 2);
(c) the pentapeptide Val-Pro-Gly-X-Gly (SEQ ID NO: 3), or VPGXG, where X is any natural or non-natural amino acid residue, and where X optionally varies among polymeric or oligomeric repeats;
(d) the pentapeptide Ala-Val-Gly-Val-Pro, or AVGVP (SEQ ID NO: 4);
(e) the pentapeptide Ile-Pro-Gly-X-Gly, or IPGXG (SEQ ID NO: 5), where X is any natural or non-natural amino acid residue, and where X optionally varies among polymeric or oligomeric repeats;
(e) the pentapeptide Ile-Pro-Gly-Val-Gly, or IPGVG (SEQ ID NO: 6);
(f) the pentapeptide Leu-Pro-Gly-X-Gly, or LPGXG (SEQ ID NO: 7), where X is any natural or non-natural amino acid residue, and where X optionally varies among polymeric or oligomeric repeats;
(g) the pentapeptide Leu-Pro-Gly-Val-Gly, or LPGVG (SEQ ID NO: 8);
(h) the hexapeptide Val-Ala-Pro-Gly-Val-Gly, or VAPGVG (SEQ ID NO: 9);
(I) the octapeptide Gly-Val-Gly-Val-Pro-Gly-Val-Gly, or GVGVPGVG (SEQ ID NO: 10);
(J) the nonapeptide Val-Pro-Gly-Phe-Gly-Val-Gly-Ala-Gly, or VPGFGVGAG (SEQ ID NO: 11); and
(K) the nonapeptides Val-Pro-Gly-Val-Gly-Val-Pro-Gly-Gly, or VPGVGVPGG (SEQ ID NO: 12).

Such structural units defined by SEQ ID NOS:1-12 may form structural repeat units, or may be used in combination to form an ELP component in accordance with the invention. In some embodiments, the ELP component is formed entirely (or almost entirely) of one or a combination of (e.g., 2, 3 or 4) structural units selected from SEQ ID NOS: 1-12. In other embodiments, at least 75%, or at least 80%, or at least 90% of the ELP component is formed from one or a combination of structural units selected from SEQ ID NOS: 1-12, and which may be present as repeating units.

In certain embodiments, the ELP component(s) contain repeat units, including tandem repeating units, of the pentapeptide Val-Pro-Gly-X-Gly (SEQ ID NO:3), where X is as defined above, and where the percentage of Val-Pro-Gly-X-Gly (SEQ ID NO:3) pentapeptide units taken with respect to the entire ELP component (which may comprise structural units other than VPGXG (SEQ ID NO:3)) is greater than about 75%, or greater than about 85%, or greater than about 95% of the ELP component. The ELP component may contain motifs having a 5 to 15-unit repeat (e.g. about 10-unit repeat) of the pentapeptide of SEQ ID NO: 3, with the guest residue X varying among at least 2 or at least 3 of the units. The guest residues may be independently selected, such as from the amino acids V, I, L, A, G, and W (and may be selected so as to retain a desired inverse phase transition property). The repeat motif itself may be repeated, for example, from about 5 to about 15 times, such as about 8 to 12 times, to create an exemplary ELP component. The ELP component as described in this paragraph may of course be constructed from any one of the structural units defined by SEQ ID NOS: 1-12, or a combination thereof.

In some embodiments, the ELP component may include a β-turn structure that provides an elastin-like property (e.g., inverse phase transition). Exemplary peptide sequences suitable for creating a β-turn structure are described in International Patent Application. PCT/US96/05186, which is hereby incorporated by reference in its entirety. For example, the fourth residue (X) in the elastin pentapeptide sequence, VPGXG (SEQ ID NO:3), can be altered without eliminating the formation of a β-turn.

In certain embodiments, the ELP components include polymeric or oligomeric repeats of the pentapeptide VPGXG (SEQ ID NO: 3), where the guest residue X is any amino acid. X may be a naturally occurring or non-naturally occurring amino acid. In some embodiments, X is selected from alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine. In some embodiments, X is a natural amino acid other than proline or cysteine.

The guest residue X (e.g., with respect to SEQ ID NO: 3, or other ELP structural unit) may be a non-classical (non-genetically encoded) amino acid. Examples of non-classical amino acids include: D-isomers of the common amino acids, 2,4-diaminobutyric acid, α-amino isobutyric acid, A-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

Selection of X is independent in each ELP structural unit (e.g., for each structural unit defined herein having a guest residue X). For example, X may be independently selected for each structural unit as an amino acid having a positively charged side chain, an amino acid having a negatively charged side chain, or an amino acid having a neutral side chain, including in some embodiments, a hydrophobic side chain.

In still other embodiments, the ELP component(s) may include polymeric or oligomeric repeats of the pentapeptides VPGXG (SEQ ID NO:3), IPGXG (SEQ ID NO:5) or LPGXG (SEQ ID NO:7), or a combination thereof, where X is as defined above.

In each embodiment, the structural units, or in some cases polymeric or oligomeric repeats, of the ELP sequences may be separated by one or more amino acid residues that do not eliminate the overall effect of the molecule, that is, in imparting certain improvements to the therapeutic component as described. In certain embodiments, such one or more amino acids also do not eliminate or substantially affect the phase transition properties of the ELP component (relative to the deletion of such one or more amino acids).

In each repeat, X is independently selected. The structure of the resulting ELP components may be described using the notation ELPk [XᵢYⱼ-n], where k designates a particular ELP repeat unit, the bracketed capital letters are single letter amino acid codes and their corresponding subscripts designate the relative ratio of each guest residue X in the structural units (where applicable), and n describes the total length of the ELP in number of the structural repeats. For example, ELP1 [V₅A₂G₃-10] designates an ELP component containing 10 repeating units of the pentapeptide VPGXG (SEQ ID NO:3), where X is valine, alanine, and glycine at a relative ratio of 5:2:3; ELP1 [K₁V₂F₁-4] designates an ELP component containing 4 repeating units of the pentapeptide VPGXG (SEQ ID NO:3), where X is lysine, valine, and phenylalanine at a relative ratio of 1:2:1; ELP1 [K₁V₇F₁-9] designates a polypeptide containing 9 repeating units of the pentapeptide VPGXG (SEQ ID NO:3), where X is lysine, valine, and phenylalanine at a relative ratio of 1:7:1; ELP1 [V-5] designates a polypeptide containing 5 repeating units of the pentapeptide VPGXG (SEQ ID NO:3), where X is exclusively valine; ELP1 [V-20] designates a polypeptide containing 20 repeating units of the pentapeptide VPGXG (SEQ ID NO:3), where X is exclusively valine; ELP2 [5] designates a polypeptide containing 5 repeating units of the pentapeptide AVGVP (SEQ ID NO:4); ELP3 [V-5] designates a polypeptide containing 5 repeating units of the pentapeptide IPGXG (SEQ ID NO:5), where X is exclusively valine; ELP4 [V-5] designates a polypeptide containing 5 repeating units of the pentapeptide LPGXG (SEQ ID NO:7), where X is exclusively valine. Such ELP components as described in this paragraph may be used in connection with the present invention to increase the therapeutic properties of the therapeutic component. Further, the relative ratios of guest residues as described in this paragraph may be used in connection with the fusion protein of the invention, and the corresponding methods, regardless of ELP size.

Further, the Tt is a function of the hydrophobicity of the guest residue. Thus, by varying the identity of the guest residue(s) and their mole fraction(s), ELPs can be synthesized that exhibit an inverse transition over a 0-100° C. range. Thus, the Tt at a given ELP length may be decreased by incorporating a larger fraction of hydrophobic guest residues in the ELP sequence. Examples of suitable hydrophobic guest residues include valine, leucine, isoleucine, phenyalanine, tryptophan and methionine. Tyrosine, which is moderately hydrophobic, may also be used. Conversely, the Tt may be increased by incorporating residues, such as those selected from the group consisting of: glutamic acid, cysteine, lysine, aspartate, alanine, asparagine, serine, threonine, glycine, arginine, and glutamine; preferably selected from alanine, serine, threonine and glutamic acid.

The ELP component in some embodiments is selected or designed to provide a Tt ranging from about 10 to about 80° C., such as from about 35 to about 60° C., or from about 38 to about 45° C. In some embodiments, the Tt is greater than about 40° C. or greater than about 42 ° C., or greater than about 45 ° C., or greater than about 50 ° C. The transition temperature, in some embodiments, is above the body temperature of the subject or patient (e.g., >37° C.) thereby remaining soluble in vivo, or in other embodiments, the Tt is below the body temperature (e.g., <37° C.) to provide alternative advantages, such as in vivo formation of a drug depot for sustained release of the therapeutic agent. See, for example, US 2007/0009602, which is hereby incorporated by reference in its entirety.

The Tt of the ELP component can be modified by varying ELP chain length, as the Tt generally increases with decreasing MW. For polypeptides having a molecular weight >100,000, the hydrophobicity scale developed by Urry et al. (PCT/US96/05186, which is hereby incorporated by reference in its entirety) provides one means for predicting the approximate Tt of a specific ELP sequence. However, in some embodiments, ELP component length can be kept relatively small, while maintaining a target Tt, by incorporating a larger fraction of hydrophobic guest residues (e.g., amino acid residues having hydrophobic side chains) in the ELP sequence. For polypeptides having a molecular weight <100,000, the Tt may be predicted or determined by the following quadratic function: Tt =M₀+M₁X+M₂X² where X is the MW of the fusion protein, and M₀=116.21; M₁=−1.7499; M₂=0.010349.

While the Tt of the ELP component, and therefore of the ELP component coupled to a therapeutic component, is affected by the identity and hydrophobicity of the guest residue, X, additional properties of the molecule may also be affected. Such properties include, but are not limited to solubility, bioavailability, persistence, half-life, potency and safety of the molecule.

As described in PCT/US2007/077767 (published as WO 2008/030968), which is hereby incorporated by reference in its entirety, the ELP-coupled PAH component can retain the PAH enzymatic activity. Additionally, ELPs themselves can exhibit long half-lives. Therefore, ELP components in accordance with the present invention substantially increase (e.g. by greater than 10, 50, 100, 500, 1000, 5000, or 10,000 times or more, in specific embodiments) the half-life of the therapeutic component when conjugated thereto. Such half-life (or in some embodiments persistance or rate of clearance) is determined in comparison to the half-life of the free (unconjugated or unfused) form of the PAH component. Furthermore, ELPs may target high blood content organs, when administered in vivo, and thus, can partition in the body, to provide a predetermined desired corporeal distribution among various organs or regions of the body, or a desired selectivity or targeting of a therapeutic agent.

The invention thus provides fusion protein agents for therapeutic (in vivo) application, where the therapeutic component (PAH) is enzymatically active. In some forms, the coupling of the therapeutic component to the ELP component is effected by direct covalent bonding or indirect (through appropriate spacer groups) bonding (as described elsewhere herein). Further, the therapeutic component(s) and the ELP component(s) can be structurally arranged in any suitable manner involving such direct or indirect covalent bonding, relative to one another.

### Positioning and Coupling of Sequences

A PAH fusion protein in accordance with the invention includes at least one heterologous component (e.g., an ELP component) and at least one PAH component, each as described above. Generally, the heterologous component and PAH components are associated with one another by genetic fusion. For example, the fusion protein may be generated by translation of a polynucleotide encoding the PAH component cloned in-frame with the heterologous component (or vice versa).

The PAH fusion protein may contain one or more copies of the PAH component attached to the N-terminus and/or the C-terminus of an ELP component, as described. In some embodiments, the PAH component is attached to both the N-terminus and C-terminus of the ELP component. Alternatively, the PAH fusion protein may contain one or more copies of an ELP component attached to the N-terminus and/or the C-terminus of the PAH component. In some embodiments, the ELP component is attached to both the N-terminus and C-terminus of the PAH component.

In certain embodiments, the ELP component and the therapeutic components can be fused using a linker peptide of various lengths to provide greater physical separation and allow more spatial mobility between the fused portions, and thus maximize the accessibility of the therapeutic component, for instance, for binding cofactor and/or substrate. The linker peptide may consist of amino acids that are flexible or more rigid. For example, a flexible linker may include amino acids having relatively small side chains, and which may be hydrophilic. Without limitation, the flexible linker may contain a stretch of glycine and/or serine residues. More rigid linkers may contain, for example, more sterically hindering amino acid side chains, such as (without limitation) tyrosine or histidine. The linker may be less than about 50, 40, 30, 20, 10, or 5 amino acid residues. The linker can be covalently linked to and between an ELP component and a therapeutic component, for example, via recombinant fusion.

The linker or peptide spacer may be protease-cleavable or non-cleavable. By way of example, cleavable peptide spacers include, without limitation, a peptide sequence recognized by proteases (in vitro or in vivo) of varying type, such as Tev, thrombin, factor Xa, plasmin (blood proteases), metalloproteases, cathepsins (e.g., GFLG, etc.), and proteases found in other corporeal compartments. In some embodiments employing cleavable linkers, the fusion protein may be inactive, less active, or less potent as a fusion, which is then activated upon cleavage of the spacer in vivo. Alternatively, where the therapeutic agent is sufficiently active as a fusion, a non-cleavable spacer may be employed. The non-cleavable spacer may be of any suitable type, including, for example, non-cleavable spacer moieties having the formula [(Gly)n-Ser]m, where n is from 1 to 4, inclusive, and m is from 1 to 4, inclusive. Alternatively, a short ELP sequence different than the backbone ELP could be employed instead of a linker or spacer, while accomplishing the necessary effect.

In still other embodiments, the therapeutic agent is a recombinant fusion having a therapeutic component flanked on each terminus by an ELP component. At least one of said ELP components may be attached via a cleavable spacer, such that the therapeutic component is inactive, but activated in vivo by proteolytic removal of a single ELP component. The resulting single ELP fusion being active, and having an enhanced half-life (or other property described herein) in vivo.

In other embodiments, the present invention provides chemical conjugates of the ELP component and the therapeutic component. The conjugates can be made by chemically coupling an ELP component to a therapeutic component by any number of methods well known in the art (See e.g. Nilsson et al., 2005, Ann Rev Biophys Bio Structure 34: 91-118). In some embodiments, the chemical conjugate can be formed by covalently linking the therapeutic component to the ELP component, directly or through a short or long linker moiety, through one or more functional groups on the therapeutic proteinacious component, e. g., amine, carboxyl, phenyl, thiol or hydroxyl groups, to form a covalent conjugate. Various conventional linkers can be used, e. g., diisocyanates, diisothiocyanates, carbodiimides, bis(hydroxysuccinimide) esters, maleimide-hydroxysuccinimide esters, glutaraldehyde and the like.

Non-peptide chemical spacers can additionally be of any suitable type, including for example, by functional linkers described in Bioconjugate Techniques, Greg T. Hermanson, published by Academic Press, Inc., 1995, and those specified in the Cross-Linking Reagents Technical Handbook, available from Pierce Biotechnology, Inc. (Rockford, Ill.), the disclosures of which are hereby incorporated by reference, in their respective entireties. Illustrative chemical spacers include homobifunctional linkers that can attach to amine groups of Lys, as well as heterobifunctional linkers that can attach to Cys at one terminus, and to Lys at the other terminus.

### Polynucleotides, Vectors, and Host Cells

In another aspect, the invention provides polynucleotides comprising a nucleotide sequence encoding the PAH fusion protein (as described). Such polynucleotides further comprise, in addition to sequences encoding the heterologous component and PAH components, one or more expression control elements. For example, the polynucleotide, may comprise one or more promoters or transcriptional enhancers, ribosomal binding sites, transcription termination signals, and polyadenylation signals, as expression control elements. The polynucleotide may be inserted within any suitable vector, including an expression vector, and which may be contained within any suitable host cell for expression. The polynucleotide may be designed for introduction and/or protein expression in any suitable host cell, including bacterial cells, yeast cells, and mammalian cells.

A vector comprising the polynucleotide can be introduced into a cell for expression of the therapeutic agent. The vector can remain episomal or become chromosomally integrated, as long as the insert encoding the therapeutic agent can be transcribed. Vectors can be constructed by standard recombinant DNA technology. Vectors can be plasmids, phages, cosmids, phagemids, viruses, or any other types known in the art, which are used for replication and expression in prokaryotic or eukaryotic cells. It will be appreciated by one of skill in the art that a wide variety of components known in the art (such as expression control elements) may be included in such vectors, including a wide variety of transcription signals, such as promoters and other sequences that regulate the binding of RNA polymerase onto the promoter. Any promoter known to be effective in the cells in which the vector will be expressed can be used to initiate expression of the therapeutic agent. Suitable promoters may be inducible or constitutive. Examples of suitable promoters include the SV40 early promoter region, the promoter contained in the 3′ long terminal repeat of Rous sarcoma virus, the HSV-1 (herpes simplex virus-1) thymidine kinase promoter, the regulatory sequences of the metallothionein gene, etc., as well as the following animal transcriptional control regions, which exhibit tissue specificity and have been utilized in transgenic animals: elastase I gene control region which is active in pancreatic acinar cells; insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in erythroid cells, myelin basic protein gene control region which is active in oligodendrocyte cells in the brain, myosin light chain-2 gene control region which is active in skeletal muscle, and gonadotropin releasing hormone gene control region which is active in the hypothalamus.

In other aspects, the invention provides methods of making the PAH fusion proteins. The method comprises introducing a polynucleotide encoding the PAH fusion protein (as described above) into a host cell suitable for expression of the fusion protein, such as E. coli, yeast, or mammalian cell line. The construction of the encoding polynucleotide for expressing the fusion protein, and its subsequent expression, may employ standard recombinant DNA and molecular cloning and protein expression techniques. Such are described, for example, in Sambrook, J., Fritsch, E. F, and Maniatis, T. Molecular Cloning: A Laboratory Manual; Cold Spring Harbor Laboratory Press: Cold Spring Harbor, 1989. The expressed protein may be recovered by any suitable method known in the art.

In some embodiments, the PAH fusion protein is purified and recovered by temperature cycling, as described in U.S. Pat. No. 6,852,834, which is hereby incorporated by reference.

In certain embodiments, the purified protein may be combined with BH4 cofactor, so as to prepare a catalytically active PAH fusion protein.

### Pharmaceutical Compositions

The present invention further provides pharmaceutical compositions comprising an effective amount of the fusion proteins of the invention (as described above) together with a pharmaceutically acceptable carrier, diluent, or excipient. Such pharmaceutical compositions are effective for treating or ameliorating hyperphenylalaninemia or PKU, as described herein.

The therapeutic agents of the invention may be administered per se as well as in various forms including pharmaceutically acceptable esters, salts, and other physiologically functional derivatives thereof. In such pharmaceutical formulations, the therapeutic agents can be used together or formulated with other therapeutic ingredients, such as the PAH cofactor BH4 or synthetic or functional version thereof (e.g., KUVAN, sapropterin hydrochloride).

The carrier(s) must be pharmaceutically acceptable in the sense of being compatible with the other ingredients of the formulation and not unduly deleterious to the recipient thereof.

The formulations of the therapeutic agent include those suitable for parenteral as well as non-parenteral administration. Exemplary administration modalities include oral, buccal, topical, nasal, subcutaneous, intramuscular, and intravenous, among others. Formulations suitable for oral and parenteral administration are preferred.

The formulations comprising the therapeutic agent of the present invention may conveniently be presented in unit dosage forms and may be prepared by any of the methods well known in the art of pharmacy. Such methods generally include the step of bringing the therapeutic agents into association with a carrier which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation.

Formulations suitable for oral administration may be presented as discrete units such as capsules, cachets, tablets, or lozenges, each containing a predetermined amount of the active ingredient as a powder or granules; or a suspension in an aqueous liquor or a non-aqueous liquid, such as a syrup, an elixir, an emulsion, or a draught. Oral formulations may be desirably administered before, during, or just after a meal, to convert dietary phenylalanine to tyrosine.

A tablet may be made by compression or molding, optionally with one or more accessory ingredients. Compressed tablets may be prepared by compressing in a suitable machine, with the therapeutic agent being in a free-flowing form such as a powder or granules which optionally is mixed with a binder, disintegrant, lubricant, inert diluent, surface active agent, or discharging agent. Molded tablets comprised of a mixture of the powdered therapeutic agents with a suitable carrier may be made by molding in a suitable machine.

A syrup may be made by adding the therapeutic agents to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration conveniently comprise a sterile aqueous preparation of the therapeutic agent, which preferably is isotonic with the blood of the recipient (e.g., physiological saline solution). Such formulations may include suspending agents and thickening agents or other microparticulate systems which are designed to target the therapeutic agent to the circulation or one or more organs. The formulations may be presented in unit-dose or multi-dose form.

In addition to the aforementioned ingredients, the formulations of this invention may further include one or more accessory ingredient(s) selected from diluents, buffers, flavoring agents, disintegrants, surface active agents, thickeners, lubricants, preservatives (including antioxidants), and the like.

While one of skill in the art can determine the desirable dose in each case (including a unit dose for depot administration), a suitable dose of the therapeutic agent for achievement of therapeutic benefit, may, for example, be in a range of about 1 microgram (μg) to about 100 milligrams (mg) per kilogram body weight of the recipient, or in a range of about 10 μg to about 50 mg per kilogram body weight, or in a range of about 10 μg to about 50 mg per kilogram body weight. The desired dose may be presented as one dose or two or more sub-doses administered at appropriate intervals throughout the dosing period (e.g., one week, two weeks, etc. . . . ). These sub-doses can be administered in unit dosage forms, for example, containing from about 10 μg to about 1000 mg, or from about 50 μg to about 500 mg, or from about 50 μg to about 250 mg of active ingredient per unit dosage form. Alternatively, if the condition of the recipient so requires, the doses may be administered as a continuous infusion.

The mode of administration and dosage forms will of course affect the therapeutic amount of the peptide active therapeutic agent that is desirable and efficacious for a given treatment application. For example, orally administered dosages can be at least twice, e.g., 2-10 times, the dosage levels used in parenteral administration methods. Depot formulations will also allow for significantly more therapeutic agent to be delivered, such that the agent will have a sustained release over time.

The features and advantages of the present invention are more fully shown with respect to the following non-limiting examples

### Methods of Treatment

The invention provides a method for treating hyperphenylalaninemia or PKU. The method comprises administering an effective amount of the PAH fusion protein of the invention to a patient in need.

Generally, the patient has a condition associated with elevated levels of phenylalanine (hyperphenylalaninemia). The patient may have PKU of varying severity, and of varying genetic origin. The patient may have any of the known mutations responsible for PKU or hyperphenylalaninemia, including those mapped to a mutant phenylalanine hydroxylase allele. The patient may be heterozygous or homozygous for dysfunctional PAH enzyme. In certain embodiments, the patient has one or more symptoms common with PKU patients, including, for example, a musty odor to the skin, hair, and urine; vomiting and diarrhea, leading to weight loss; irritability; skin problems, such as dry skin, or itchy skin rashes (eczema); and sensitivity to light (photosensitivity).

In certain embodiments, the patient is non-responsive or has little response to treatment with KUVAN™ (sapropterin hydrochloride). For example, the patient may continue to experience elevated levels of phenylalanine even with cofactor supplementation.

In certain embodiments, the PAH fusion protein is administered parenterally, such as by subcutaneous or intramuscular injection. The administration may be a unit dose of the PAH fusion protein as described herein. In such embodiments, the PAH fusion protein is effective for converting phenylalanine levels in the blood to tyrosine, and may employ endogenous BH4 cofactor, that is without cofactor supplementation. In other embodiments, the patient also receives cofactor supplementation therapy, such as therapy with sapropterin hydrochloride or similar agent.

The PAH fusion protein, when administered parenterally, may be administered one or more times per day (e.g., 1, 2, or 3), or once or twice per week, or from once to five times per month. In these embodiments, the PAH fusion protein may be administered as a soluble fusion protein, that persists in the circulation, as described herein, to provide sustained enzymatic activity with relatively infrequent administration. Alternatively, the PAH fusion protein is administered as a drug depot, as also described herein, to provide a sustained release of fusion protein into the circulation over time. See US 2007/0009602, which is hereby incorporated by reference.

The PAH fusion protein may alternatively be formulated and administered as an oral therapy. In such embodiments, the PAH fusion is preferably formulated with cofactor, and administered just before, during, or just after a meal, such that dietary phenylalanine may be converted to tyrosine before adsorption through the intestinal mucosa.

### EXAMPLES

### Example 1

### N-Terminal Fusion with Residues 103-428 of Human PAH

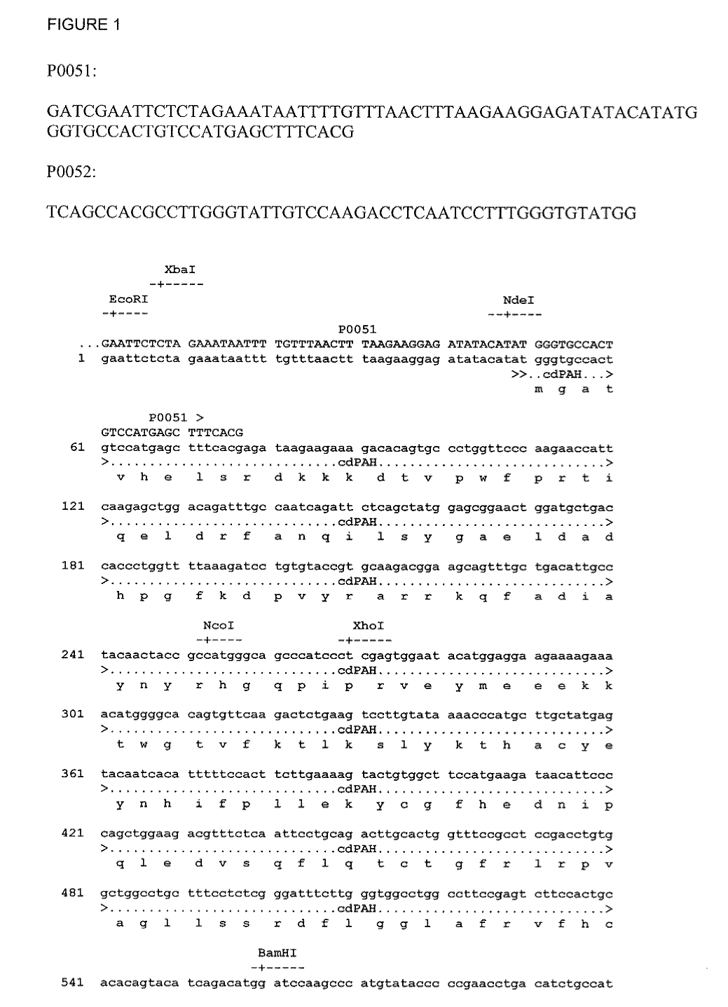

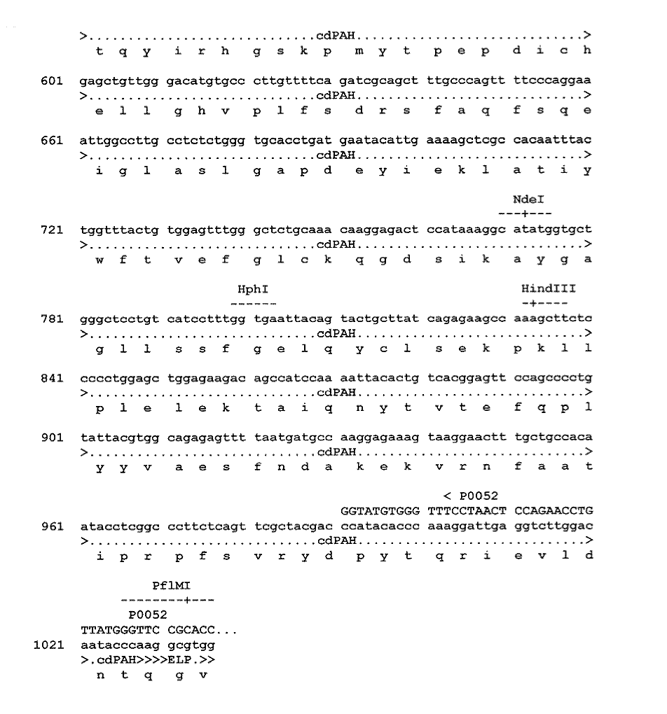

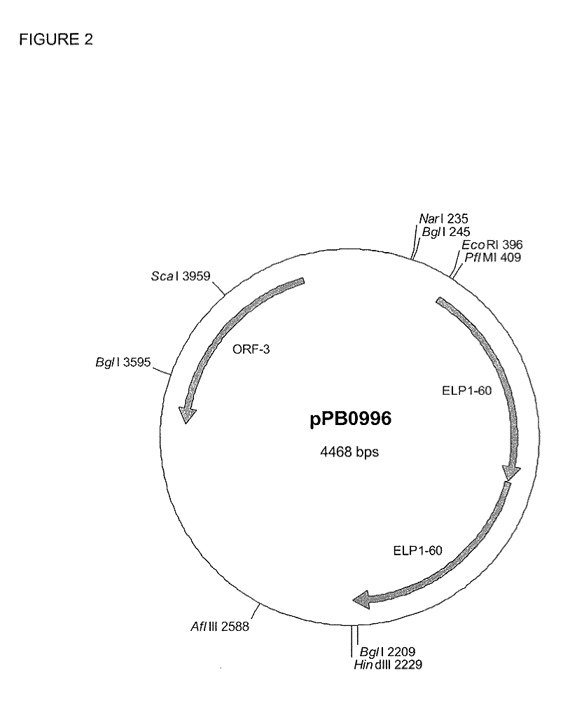

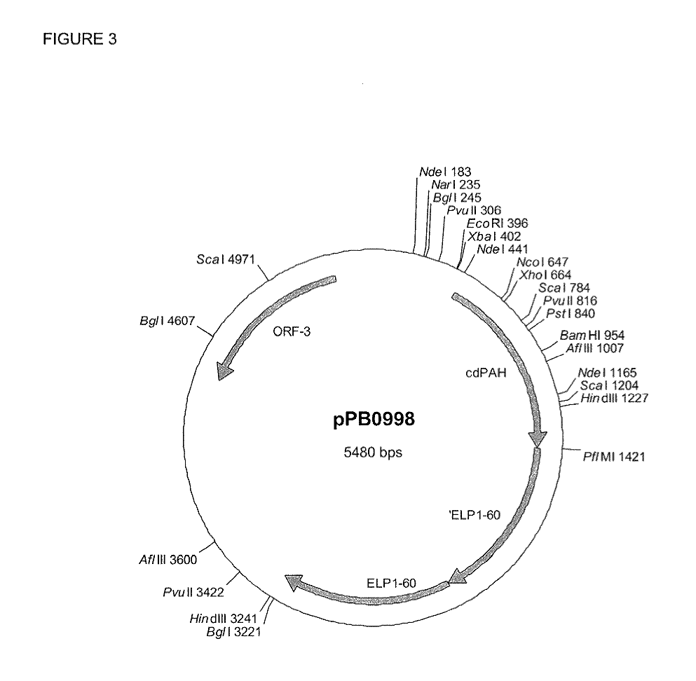

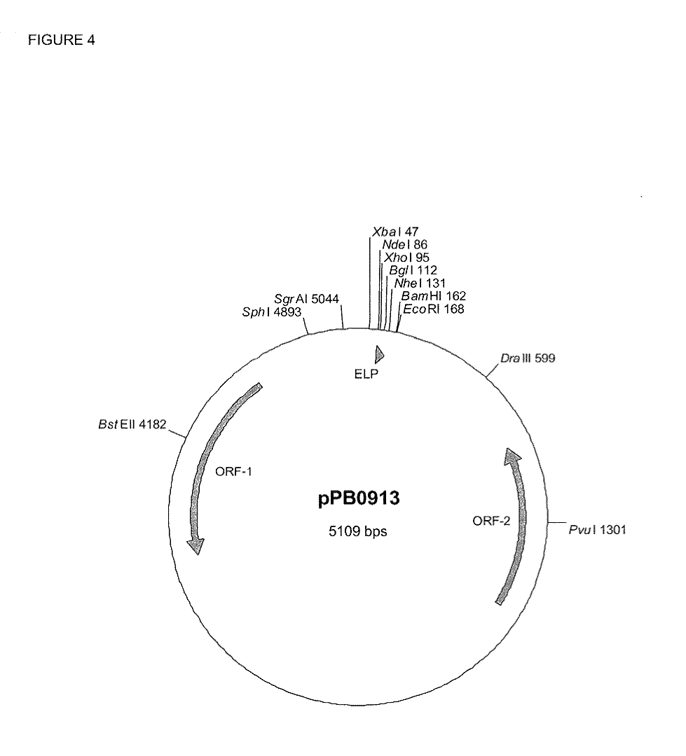

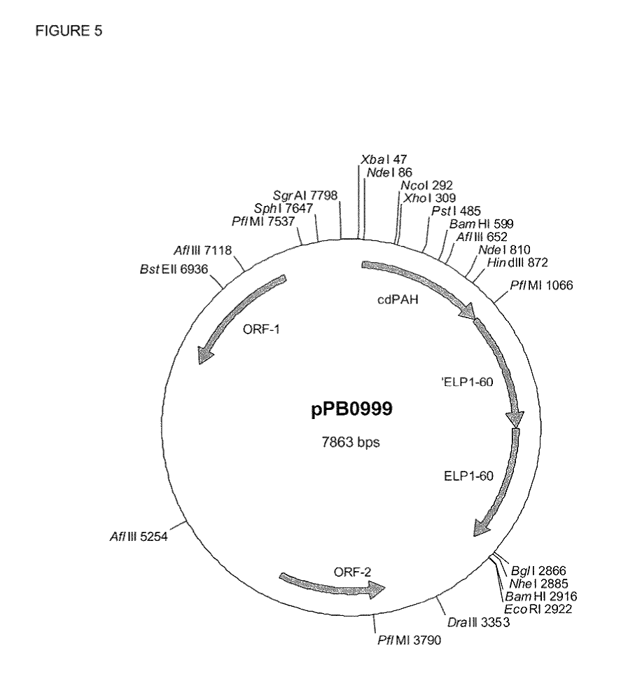

The core catalytic domain of Human PAH (cdPAH or PAH 103-428) was synthesized by PCR from a cDNA clone (OriGene SC120014) with primers P0051 and P0052. This introduces modifications at the 5′ and 3′ ends for subsequent cloning steps. FIG. 1. The resulting PCR product was digested with the restriction enzymes EcoRI and PflMI and cloned into pPB0996 (ELP1-120) (FIG. 2), which had been digested with the same restriction enzymes to give pPB0998 (FIG. 3). The insert was DNA sequenced to confirm and check for any PCR induced errors. The PAH ELP1-120 expression cassette was recovered from pPB0998 by digestion with the restriction enzymes Xbal and BglI and ligated into pPB0913 (FIG. 4) digested with the same restriction enzymes to give the final pET based construct, pPB0999 (FIG. 5). This cloning results in the first VPGXG repeat (SEQ ID NO: 3) being truncated to GVG.

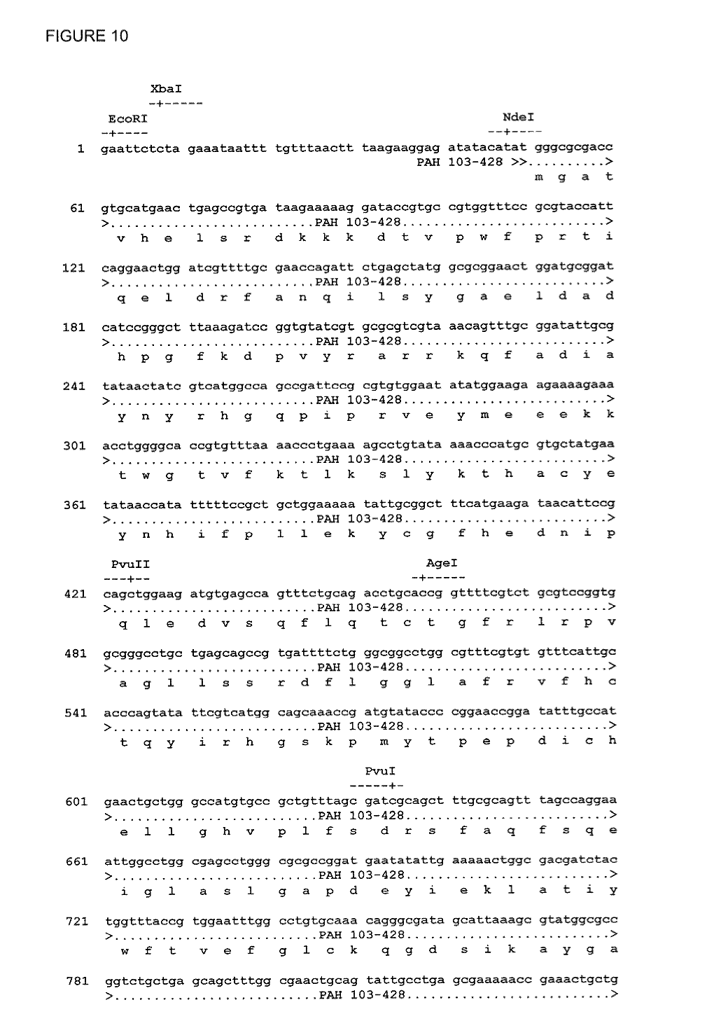

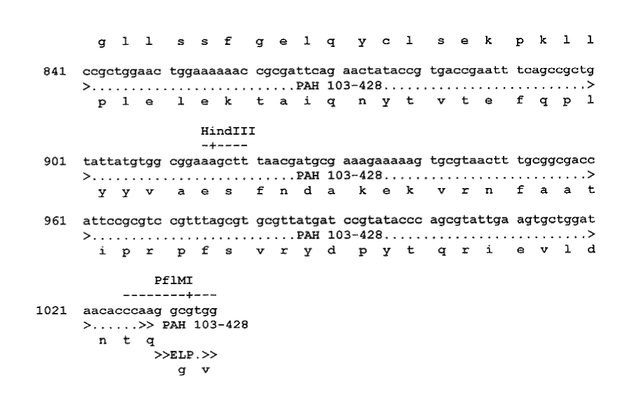

The DNA sequence for PAH was optimized for E. coil expression by selection for codon usage, mRNA secondary structure, balancing of GC content, and removal of repetitive elements where possible. The resulting sequence was then chemically synthesized. FIG. 10. Cloning was as described above.

### Example 2

### C-Terminal Fusion with Residues 103-427 of Human PAH

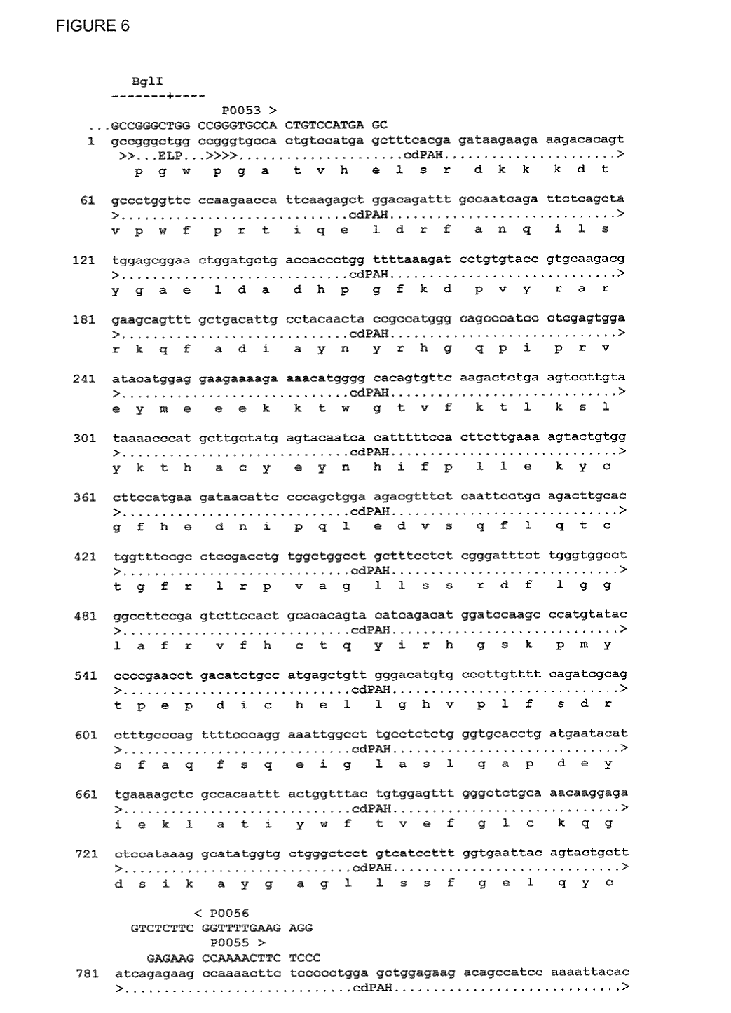

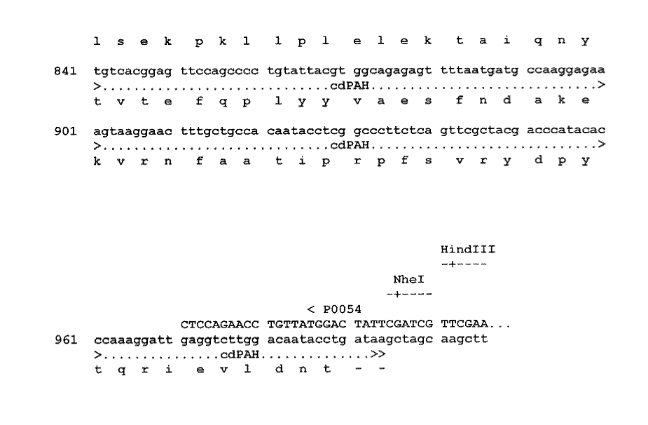

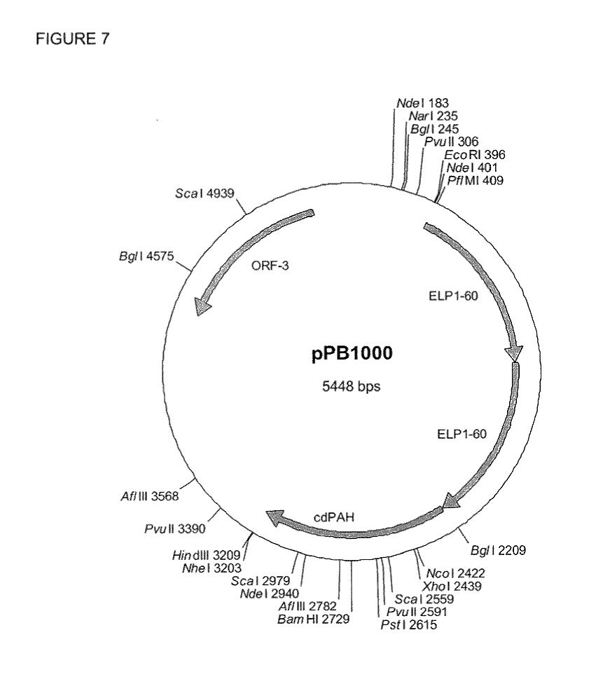

The core catalytic domain of Human PAH (cdPAH or PAH 103-427) was synthesized by PCR from a cDNA clone (OriGene SC120014) with primer pairs P0053+P0056 or P0054+P0055 to create two PCR products. The resulting PCR products were joined together by PCR with just the outer primers P0053 and P0054. This removes an internal HindIII site to enable the use of this restriction site at the subsequent cloning step. FIG. 6. The final PCR product was digested with the restriction enzymes BglI and HindIII and ligated into pPB0996 (ELP1-120), which had been digested with the same restriction enzymes to give pPB1000 (FIG. 7).

(SEQ ID NO: 19)

P0053: GTCAGCCGGGCTGGCCGGGTGCCACTGTCCATGAGC

(SEQ ID NO: 20)

P0054: GTCAAAGCTTGCTAGCTTATCAGGTATTGTCCAAGACCTC

(SEQ ID NO: 21)

P0055: GAGAAGCCAAAACTTCTCCC

(SEQ ID NO: 22)

P0056: GGAGAAGTTTTGGCTTCTCTG

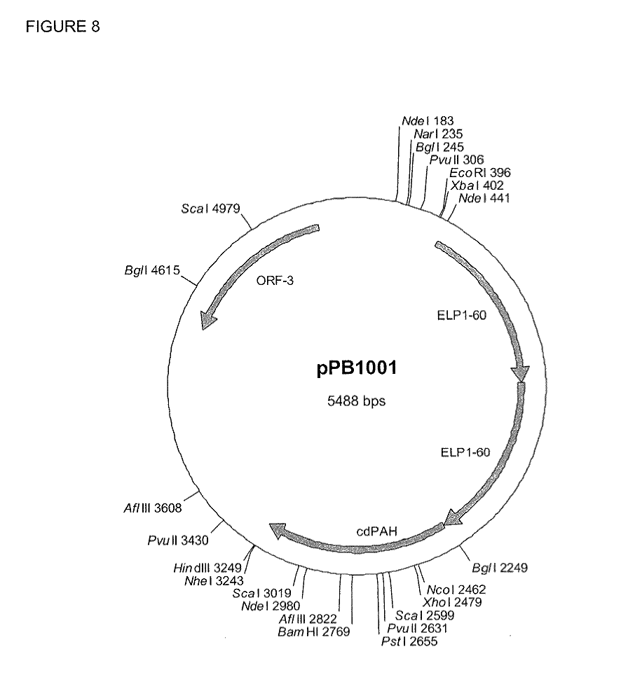

To modify the 5′ end of the expression cassette to give the correct start (MVPGVG . . . ) the plasmid pPB1000 was digested with the restriction enzymes EcoRI/PflMI and a linker created from annealing together primers P0049 and P0050 was ligated in to give the plasmid pPB1001 (FIG. 8):

```
P0049:
(SEQ ID NO: 26)
AATTCTCTAGAAATAATTTTGTTTAACTTTAAGAAGGAGATATACATATGGTTCCGGGC
P0050:
(SEQ ID NO: 27)
CGGAACCATATGTATATCTCCTTCTTAAAGTTAAACAAAATTATTTCTAGAG
                    XbaI NdeI
                -+----- P0049 --+---
1 aattctctag aaataatttt gtttaacttt aagaaggaga tatacatatg gttccgggc
            gagatc tttattaaaa caaattgaaa ttcttcctct atatgtatac caaggc
                                                          P0050 >>.ELP..>>
                                                                                                              m v p
```

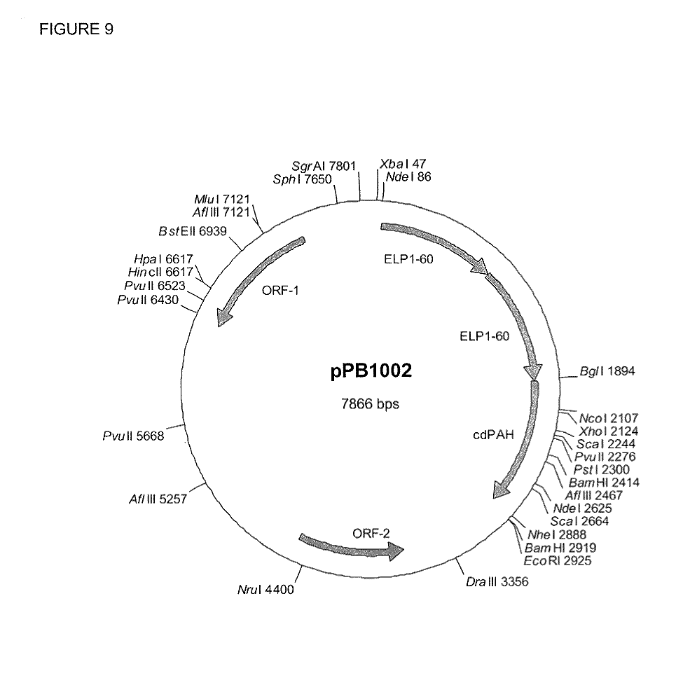

The resulting expression cassette was recovered form pPB1001 by digestion with the restriction enzymes Xbal and Nhel and ligated into pPB0913 digested with the same restriction enzymes to create the final pET based plasmid, pPB1002 (FIG. 9).

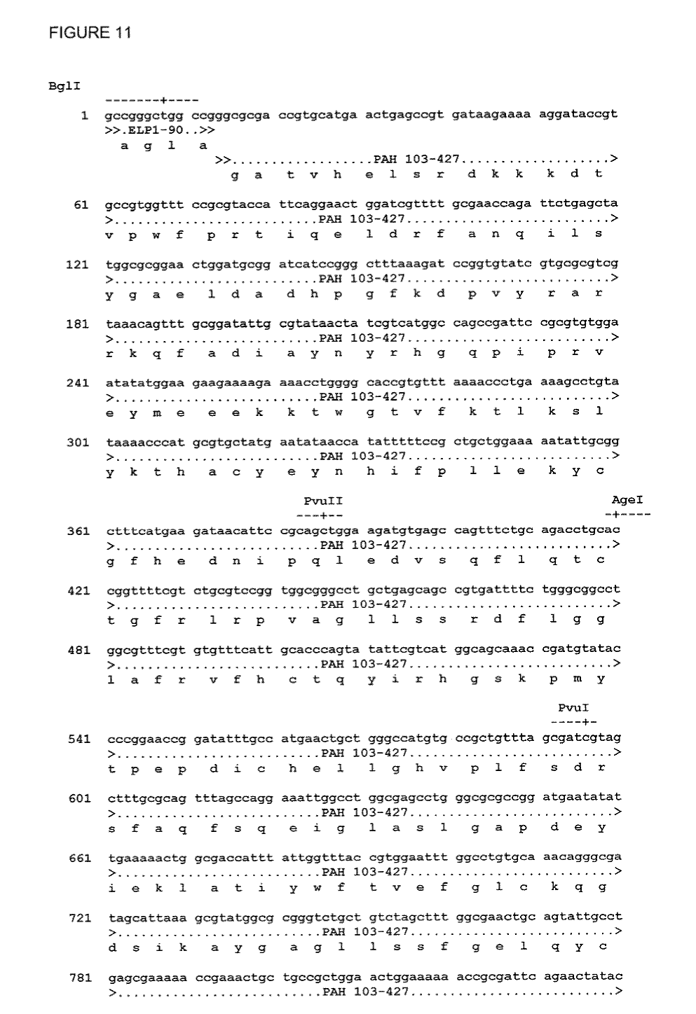

The DNA sequence for PAH was optimized for E. coli expression by selection for codon usage, mRNA secondary structure, balancing of GC content, and removal of repetitive elements where possible. FIG. 11. The resulting sequence was then chemically synthesized. Cloning of PCR product was as described above.

### Example 3

### Expression and Enzymatic Activity of PAH-ELP Fusion

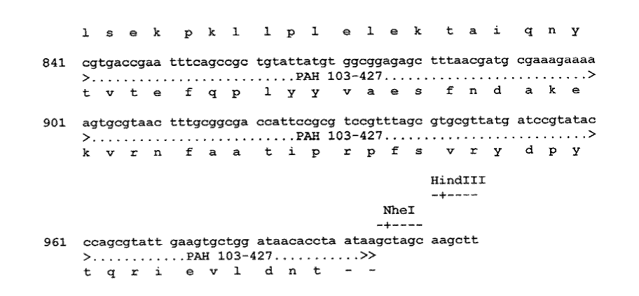

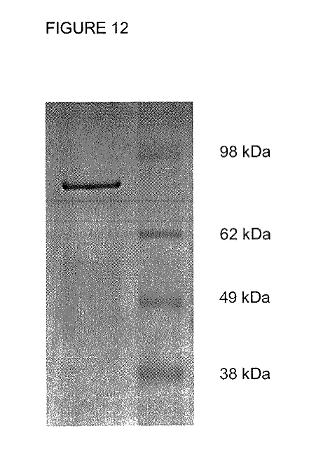

PAH (103-428)-ELP1-120 (designated PB0999) was expressed in E. coli and subsequently purified by temperature cycling. The expected molecular weight of 85 kDa as shown by SDS-PAGE (denaturing, non-reducing) was obtained. see FIG. 12.

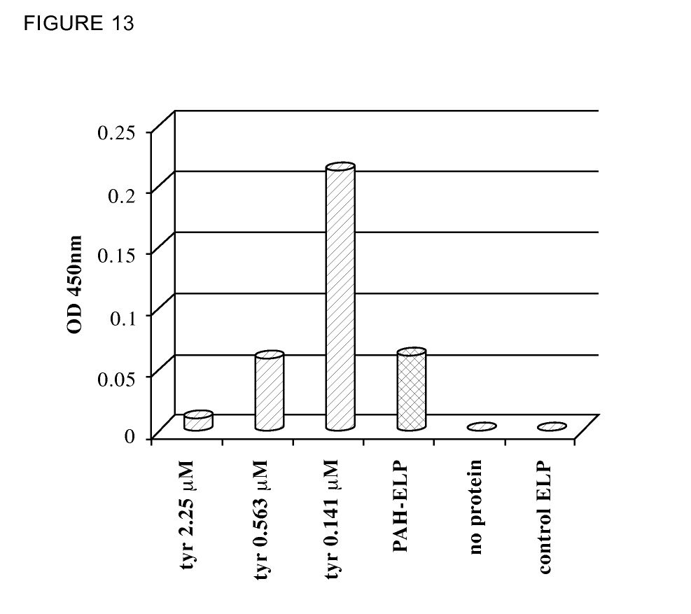

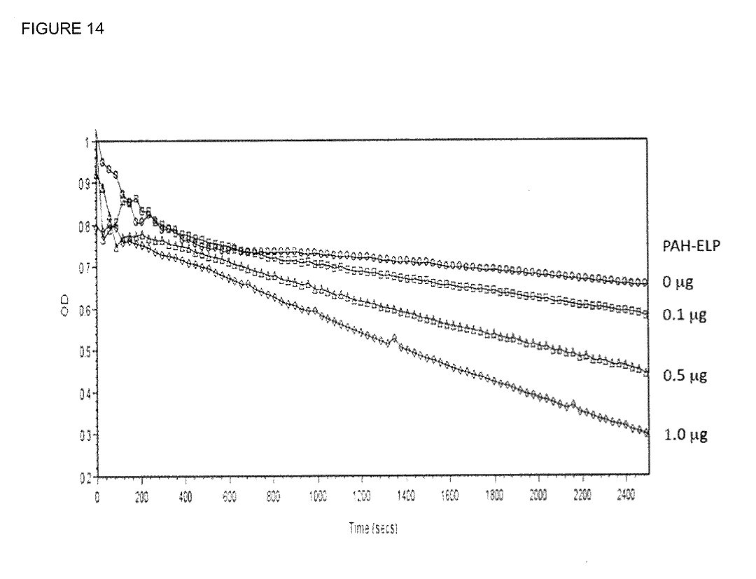

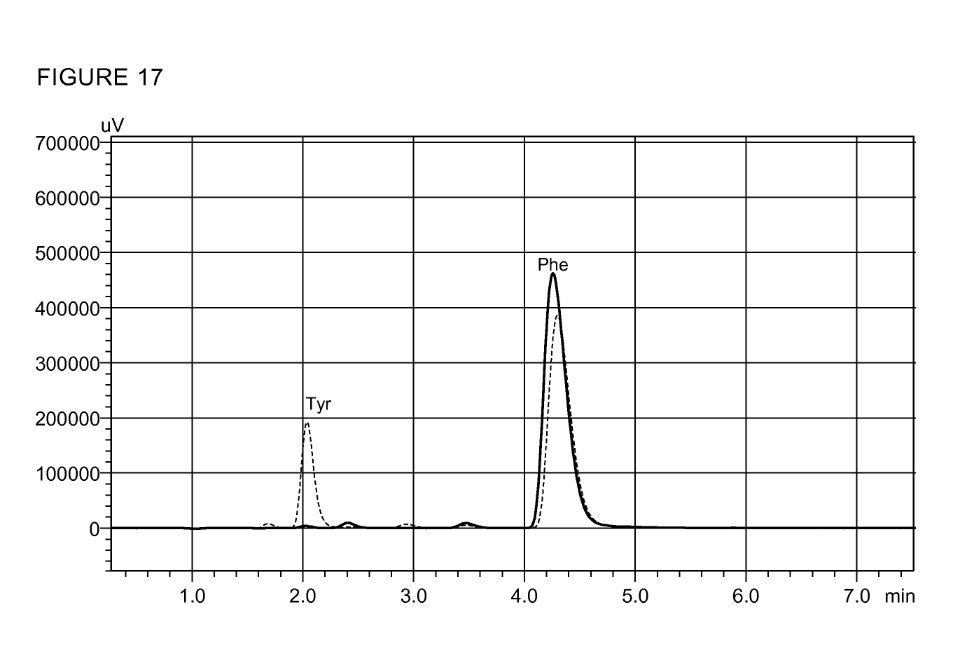

PB0999 was tested for enzymatic activity. Conversion of phenylalanine to tryrosine by PB0999 was detected by OD450 nm (FIG. 13), as well as by phenylalanine-dependent oxidation of NADH (OD 340 nm). see Macdonald et al. (1990), PNAS 87, 1965-1967. (FIG. 14). Conversion of phenylalanine to tyrosine was also determined by RP-HPLC (Shimadzu C18 column) (FIG. 17).

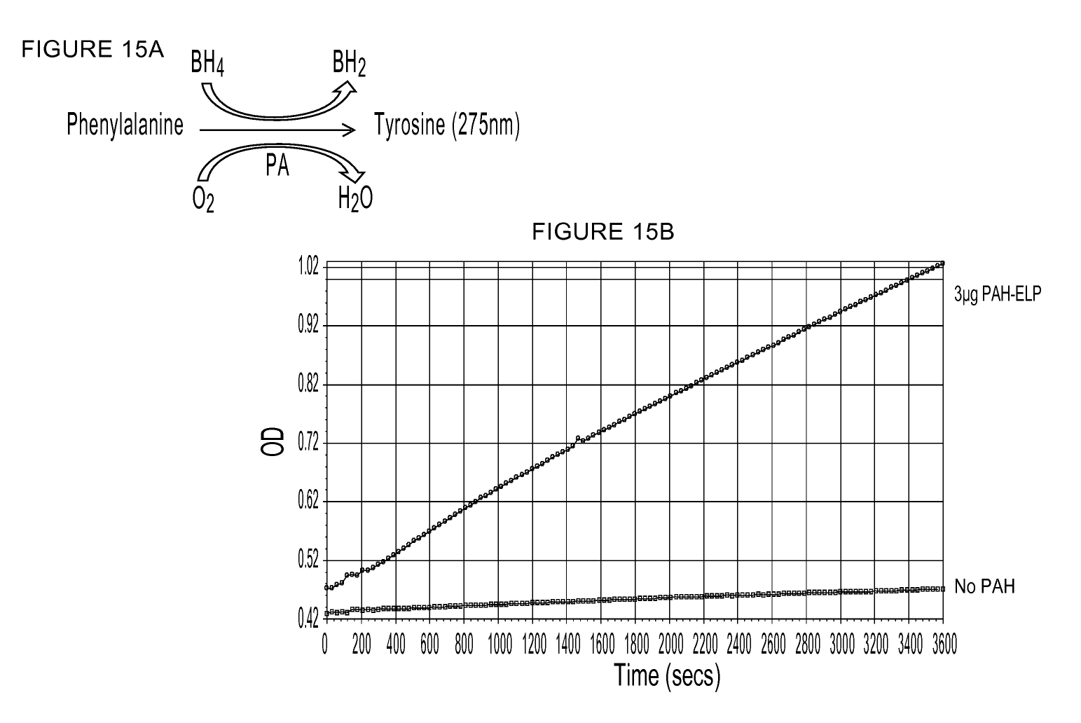

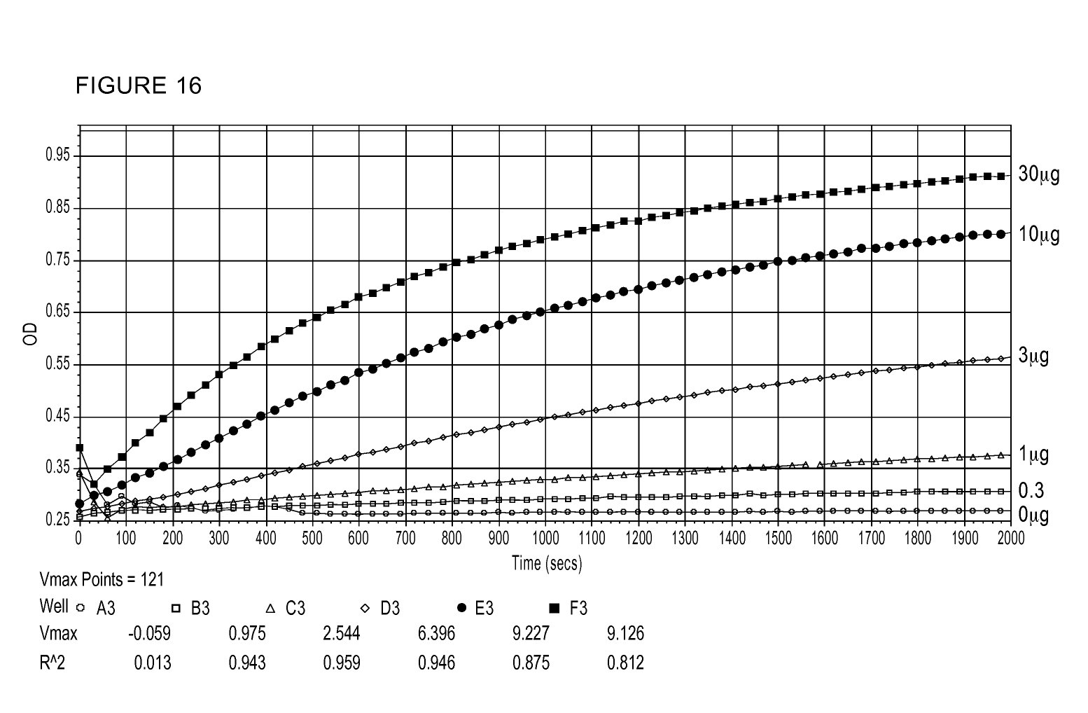

Presence of tyrosine may be determined by increase in OD at 275nm. As shown in FIG. 15B, PAH-ELP converts phenylalanine to tyrosine, as determined by an increase in OD₂₇₅. The PAH-ELP comprises PAH(103-428) with 120 pentamer ELP repeats, and exhibits a specific activity of 878 nmol tyrosine/min·mg. Compare with 1200-1502 nmol tyrosine/min·mg for PAH(103-428) as reported by Knappskog et al., Structure/function relationships in human phenylalanine hydroxylase. Eur. J. Biochem. 242:813-821 (1996). Tyrosine production is dose dependent. FIG. 16.

### Example 4

### Effect of PAH-ELP on Phenylalanine Levels in Mice

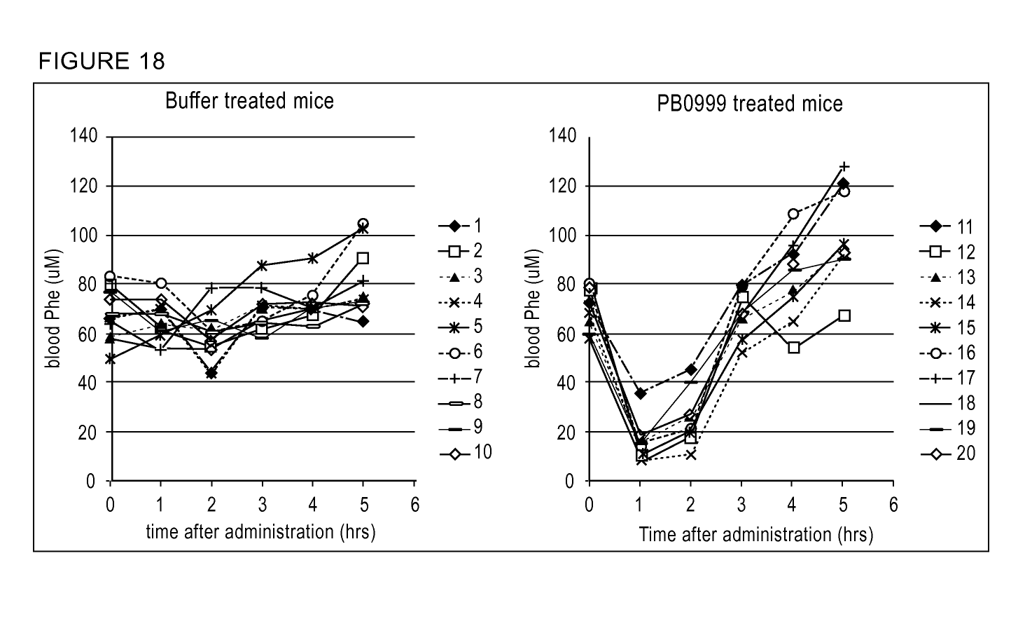

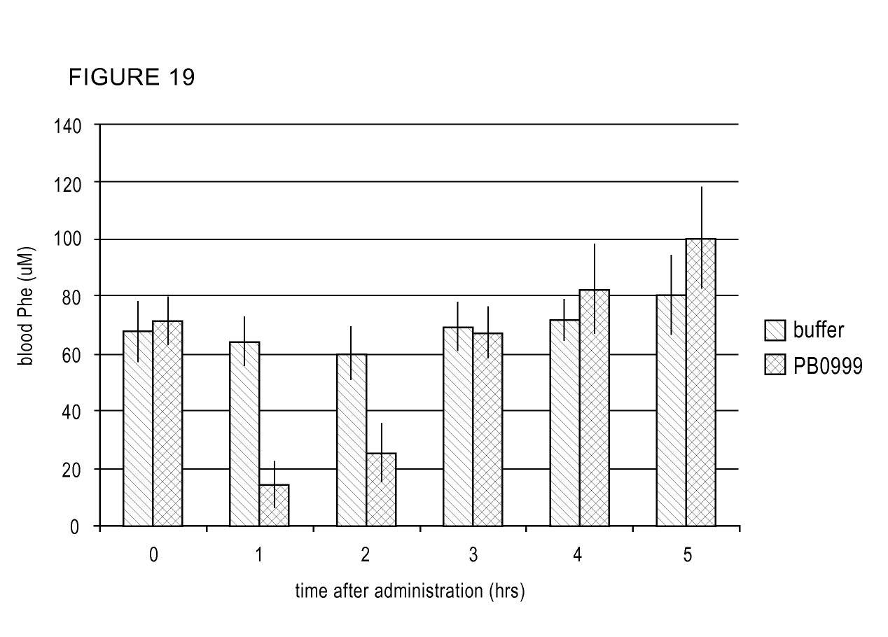

This example demonstrates that PAH (103-428)-ELP1-120 (designated PB0999) can significantly reduce levels of phenylalanine in vivo. In this experiment, mice were treated with buffer alone or PB0999, and blood phenylalanine levels were measured by RP-HPLC. Mice (n =10 per group) were injected intraperitoneally with 3 mg of either PB0999 or control buffer. As shown in FIG. 18, mice treated with PB0999 showed reduced levels of blood phenylalanine 1-2 hrs after administration of the compound. FIG. 19 reflects the average levels of blood phenylalanine among the two groups of mice. As FIG. 19 demonstrates, administration of PB0999 had a significant effect on phenylalanine levels in vivo. The average level of phenylalanine in mice treated with PB0999 was less than 20 μM after one hour of administration. In contrast, phenylalanine levels in buffer treated mice were more than 3-fold higher (>60 uM). These data suggest that the PAH fusion proteins have utility for enzyme replacement therapy in PKU patients by converting phenylalanine in the circulation to tyrosine, thereby controlling phenylalanine levels.

1. A therapeutic agent comprising a catalytic core of Phenylalanine Hydroxylase, and a heterologous mammalian protein or derivative thereof, to thereby extend half-life of the therapeutic agent.

2. The therapeutic agent of claim 1, wherein the heterologous protein or derivative is an Elastin-Like Protein (ELP) component.

3. The therapeutic agent of claim 1, wherein the catalytic core of Phenylalanine Hydroxylase comprises an amino acid sequence beginning at an amino acid within residues 80 to 115 of SEQ ID NO:24 and terminating at an amino acid within residues 400 to 450 of SEQ ID NO:24.

4. The therapeutic agent of claim 3, wherein the catalytic core of Phenylalanine Hydroxylase comprises an amino acid sequence beginning at an amino acid within residues 90 to 115 of SEQ ID NO:24.

5. The therapeutic agent of claim 3, wherein the catalytic core of Phenylalanine Hydroxylase comprises an amino acid sequence beginning at an amino acid within residues 95 to 115 of SEQ ID NO:24.

6. The therapeutic agent of claim 3, wherein the catalytic core of Phenylalanine Hydroxylase comprises an amino acid sequence beginning at an amino acid within residues 100 to 115 of SEQ ID NO:24.

7. The therapeutic agent of claim 3, wherein the catalytic core of Phenylalanine Hydroxylase comprises an amino acid sequence beginning at about amino acid 103 of SEQ ID NO:24.

8. The therapeutic agent of claim 1, wherein the catalytic core of Phenylalanine Hydroxylase comprises an amino acid sequence terminating at an amino acid within residues 400 to 430 of SEQ ID NO:24.

9. The therapeutic agent of claim 8, wherein the catalytic core of Phenylalanine Hydroxylase comprises an amino acid sequence terminating at about amino acid 428 of SEQ ID NO:24.

10. The therapeutic agent of claim 1, wherein the catalytic core of Phenylalanine Hydroxylase comprises amino acids 103 to 428 of SEQ ID NO:24.

11. The therapeutic agent of claim 1, wherein the catalytic core of Phenylalanine Hydroxylase consists essentially of amino acids 103 to 428 of SEQ ID NO:24.

12. The therapeutic agent of claim 1, wherein the catalytic core of Phenylalanine Hydroxylase consists of amino acids 103 to 428 of SEQ ID NO:24.

13. The therapeutic agent of claim 1, wherein the catalytic core of Phenylalanine Hydroxylase contains from 1 to 10 amino acid substitutions, insertions, and/or deletions with respect to SEQ ID NO:25.

14. The therapeutic agent of claim 1, wherein the catalytic core of Phenylalanine Hydroxylase is at least about 70% identical to SEQ ID NO:25.

15. The therapeutic agent of claim 1, wherein an ELP component is covalently bonded to the catalytic core of Phenylalanine Hydroxylase at an N- and/or C-terminus thereof.

16. The therapeutic agent of claim 15, wherein a first catalytic core of Phenylalanine Hydroxylase is covalently bonded to the ELP component at the N-terminus of the ELP component, and a second catalytic core of Phenylalanine Hydroxylase is covalently bonded to the ELP component at the C-terminus of the ELP component.

17. The therapeutic agent of claim 1, wherein a catalytic core of Phenylalanine Hydroxylase is covalently bonded to the ELP component at an N- and/or C-terminus thereof.

18. The therapeutic agent of claim 17, wherein a first ELP component is covalently bonded to the catalytic core pf Phenylalanine Hydroxylase at the N-terminus of the catalytic core, and a second ELP component is covalently bonded to the catalytic core of Phenylalanine Hydroxylase at the C-terminus of the catalytic core.

19. The therapeutic agent of claim 1, further comprising at least one spacer moiety between the ELP component and the catalytic core of Phenylalanine Hydroxylase.

20. The therapeutic agent of claim 19, wherein the spacer moiety comprises one or more of a protease-resistant moiety, a non-peptide chemical moiety, and a protease cleavage site.

21. The therapeutic agent of claim 20, wherein the protease cleavage site is a thrombin cleavage site, a factor Xa cleavage site, a metalloprotease cleavage site, an enterokinase cleavage site, a Tev cleavage site, and a cathepsin cleavage site.

22. The therapeutic agent of claim 1, wherein the ELP component comprises at least one repeating unit selected from SEQ ID NOS: 1-12.

23. The therapeutic agent of claim 22, wherein said repeating unit is VPGXG (SEQ ID NO: 3).

24. The therapeutic agent of claim 23, wherein X is any natural or non-natural amino acid residue, and wherein X varies among at least two units.

25. The therapeutic agent of claim 24, wherein each X is independently selected from alanine, arginine, asparagine, aspartic acid, glutamic acid, glutamine, glycine, histidine, isoleucine, leucine, lysine, methionine, phenylalanine, serine, threonine, tryptophan, tyrosine and valine residues.

26. The therapeutic agent of claim 1, wherein the ELP contains from about 60 to about 150 ELP pentamer units.

27. The therapeutic agent of claim 26, wherein the ELP contains from about 90 to about 120 ELP pentamer units.

28. The therapeutic agent of claim 1, wherein the ELP component has a Tt greater than 37° C.

29. The therapeutic agent of claim 1, wherein the therapeutic agent is a genetically encoded fusion protein.

30. A polynucleotide comprising a nucleotide sequence encoding the therapeutic agent of claim 29.

31. (canceled)

32. (canceled)

33. A vector comprising the polynucleotide of claim 30.

34. An isolated host cell containing the vector of claim 33.

35. A pharmaceutical composition comprising an effective amount of the therapeutic agent of claim 1, and a pharmaceutically acceptable carrier and/or excipient.

36. (canceled)

37. (canceled)

38. (canceled)

39. (canceled)

40. (canceled)

41. (Canceled)

42. A method for treating hyperphenylalaninemia in a subject, comprising administering an effective amount of the therapeutic agent of claim 1 to a subject in need thereof.

43. (canceled)

44. (canceled)

45. (canceled)

46. (canceled)

47. (canceled)


SEQUENCE LISTING (31 sequences)

SEQ ID NO: 1 - protein, 4 aa, Unknown
  ELP tetratpeptide
VPGG

SEQ ID NO: 2 - protein, 4 aa, Unknown
  ELP tetrapeptide
IPGG

SEQ ID NO: 3 - protein, 5 aa, Unknown
  ELP pentapeptide
VPGXG

SEQ ID NO: 4 - protein, 5 aa, Unknown
  ELP pentapeptide
AVGVP

SEQ ID NO: 5 - protein, 5 aa, Unknown
  ELP pentapeptide
IPGXG

SEQ ID NO: 6 - protein, 5 aa, Unknown
  ELP pentapeptide
IPGVG

SEQ ID NO: 7 - protein, 5 aa, Unknown
  ELP pentapeptide
LPGXG

SEQ ID NO: 8 - protein, 5 aa, Unknown
  ELP pentapeptide
LPGVG

SEQ ID NO: 9 - protein, 6 aa, Unknown
  ELP hexapeptide
VAPGVG

SEQ ID NO: 10 - protein, 8 aa, Unknown
  ELP octapeptide
GVGVPGVG

SEQ ID NO: 11 - protein, 9 aa, Unknown
  ELP nonapeptide
VPGFGVGAG

SEQ ID NO: 12 - protein, 9 aa, Unknown
  ELP nonapeptides
VPGVGVPGG

SEQ ID NO: 13 - DNA, 1036 bp, Artificial Sequence
  cdPAH-ELP fusion sequence
gaattctctagaaataattttgtttaactttaagaaggagatatacatatgggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccaaggcgtgg

SEQ ID NO: 14 - protein, 329 aa, Artificial sequence
  cdPAH-ELP fusion sequence
MGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQGV

SEQ ID NO: 15 - DNA, 81 bp, Artificial Sequence
  PCR primer P0051
gatcgaattctctagaaataattttgtttaactttaagaaggagatatacatatgggtgccactgtccatgagctttcacg

SEQ ID NO: 16 - DNA, 50 bp, Artificial Sequence
  PCR primer P0052
tcagccacgccttgggtattgtccaagacctcaatcctttgggtgtatgg

SEQ ID NO: 17 - DNA, 1006 bp, Artificial Sequence
  ELP-cdPAH fusion sequence
gccgggctggccgggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaaacttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacctgataagctagcaagctt

SEQ ID NO: 18 - protein, 329 aa, Artificial Sequence
  ELP-cdPAH fusion sequence
PGWPGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNT

SEQ ID NO: 19 - DNA, 36 bp, Artificial Sequence
  PCR primer P0053
gtcagccgggctggccgggtgccactgtccatgagc

SEQ ID NO: 20 - DNA, 40 bp, Artificial Sequence
  PCR primer P0054
gtcaaagcttgctagcttatcaggtattgtccaagacctc

SEQ ID NO: 21 - DNA, 20 bp, Artificial Sequence
  PCR primer P0055
gagaagccaaaacttctccc

SEQ ID NO: 22 - DNA, 21 bp, Artificial Sequence
  PCR primer P0056
ggagaagttttggcttctctg

SEQ ID NO: 23 - DNA, 2680 bp, Homo sapiens
cagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaagcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaaagccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagttaaatcagtaatctgtcgcatttcatcaagattaattaaaatttgggacctgcttcattcaagcttcatatatgctttgcagagaactcataaaggagcatataaggctaaatgtaaaacacaagactgtcattagaattgaattattgggcttaatataaatcgtaacctatgaagtttattttctattttagttaactatgattccaattactactttgttattgtacctaagtaaattttctttaggtcagaagcccattaaaatagttacaagcattgaacttctttagtattatattaatataaaaacatttttgtatgttttattgtaatcataaatactgctgtataaggtaataaaactctgcacctaatccccataacttccagtatcattttccaattaattatcaagtctgttttgggaaacactttgaggacatttatgatgcagcagatgttgactaaaggcttggttggtagatattcaggaaatgttcactgaataaataagtaaatacattattgaaaagcaaatctgtataaatgtgaaatttttatttgtattagtaataaaacattagtagttta

SEQ ID NO: 24 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 25 - protein, 327 aa, Homo sapiens
MGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQ

SEQ ID NO: 26 - DNA, 59 bp, Artificial Sequence
  PCR primer P0049
aattctctagaaataattttgtttaactttaagaaggagatatacatatggttccgggc

SEQ ID NO: 27 - DNA, 52 bp, Artificial Sequence
  PCR primer P0050
cggaaccatatgtatatctccttcttaaagttaaacaaaattatttctagag

SEQ ID NO: 28 - DNA, 1036 bp, Artificial Sequence
  cdPAH-ELP fusion sequence
gaattctctagaaataattttgtttaactttaagaaggagatatacatatgggcgcgaccgtgcatgaactgagccgtgataagaaaaaggataccgtgccgtggtttccgcgtaccattcaggaactggatcgttttgcgaaccagattctgagctatggcgcggaactggatgcggatcatccgggctttaaagatccggtgtatcgtgcgcgtcgtaaacagtttgcggatattgcgtataactatcgtcatggccagccgattccgcgtgtggaatatatggaagaagaaaagaaaacctggggcaccgtgtttaaaaccctgaaaagcctgtataaaacccatgcgtgctatgaatataaccatatttttccgctgctggaaaaatattgcggctttcatgaagataacattccgcagctggaagatgtgagccagtttctgcagacctgcaccggttttcgtctgcgtccggtggcgggcctgctgagcagccgtgattttctgggcggcctggcgtttcgtgtgtttcattgcacccagtatattcgtcatggcagcaaaccgatgtataccccggaaccggatatttgccatgaactgctgggccatgtgccgctgtttagcgatcgcagctttgcgcagtttagccaggaaattggcctggcgagcctgggcgcgccggatgaatatattgaaaaactggcgacgatctactggtttaccgtggaatttggcctgtgcaaacagggcgatagcattaaagcgtatggcgccggtctgctgagcagctttggcgaactgcagtattgcctgagcgaaaaaccgaaactgctgccgctggaactggaaaaaaccgcgattcagaactataccgtgaccgaatttcagccgctgtattatgtggcggaaagctttaacgatgcgaaagaaaaagtgcgtaactttgcggcgaccattccgcgtccgtttagcgtgcgttatgatccgtatacccagcgtattgaagtgctggataacacccaaggcgtgg

SEQ ID NO: 29 - DNA, 1006 bp, Artificial Sequence
  ELP-cdPAH fusion sequence
gccgggctggccgggcgcgaccgtgcatgaactgagccgtgataagaaaaaggataccgtgccgtggtttccgcgtaccattcaggaactggatcgttttgcgaaccagattctgagctatggcgcggaactggatgcggatcatccgggctttaaagatccggtgtatcgtgcgcgtcgtaaacagtttgcggatattgcgtataactatcgtcatggccagccgattccgcgtgtggaatatatggaagaagaaaagaaaacctggggcaccgtgtttaaaaccctgaaaagcctgtataaaacccatgcgtgctatgaatataaccatatttttccgctgctggaaaaatattgcggctttcatgaagataacattccgcagctggaagatgtgagccagtttctgcagacctgcaccggttttcgtctgcgtccggtggcgggcctgctgagcagccgtgattttctgggcggcctggcgtttcgtgtgtttcattgcacccagtatattcgtcatggcagcaaaccgatgtataccccggaaccggatatttgccatgaactgctgggccatgtgccgctgtttagcgatcgtagctttgcgcagtttagccaggaaattggcctggcgagcctgggcgcgccggatgaatatattgaaaaactggcgaccatttattggtttaccgtggaatttggcctgtgcaaacagggcgatagcattaaagcgtatggcgcgggtctgctgtctagctttggcgaactgcagtattgcctgagcgaaaaaccgaaactgctgccgctggaactggaaaaaaccgcgattcagaactataccgtgaccgaatttcagccgctgtattatgtggcggagagctttaacgatgcgaaagaaaaagtgcgtaactttgcggcgaccattccgcgtccgtttagcgtgcgttatgatccgtatacccagcgtattgaagtgctggataacacctaataagctagcaagctt

SEQ ID NO: 30 - protein, 329 aa, Artificial Sequence
  ELP-cdPAH fusion sequence
AGLAGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNT

SEQ ID NO: 31 - protein, 6 aa, Artificial Sequence
  Encoded sequence of pBP1001 modified start sequence
MVPGVG